(19) United States
(12) Patent Application Publication
(10) Pub. No.: US 2005/0124947 A1
(43) Pub. Date: Jun. 9, 2005

(54) Absorbent Article With Indicator Device

(75) Inventor: Ingemar Fernfors, Molndal (SE)

Correspondence Address: BURNS DOANE SWECKER & MATHIS L L P, POST OFFICE BOX 1404, ALEXANDRIA, VA, 22313-1404, US

(73) Assignee: SCA HYGIENE PRODUCTS AB, Goteborg (SE)

(21) Appl. No.: 10/904,646

(22) Filed: Nov. 20, 2004

Related U.S. Application Data
(60) Provisional application No. 60/524,211, filed on Nov. 24, 2003.

(51) Int. Cl. A61F 13/15; A61F 13/20

(52) U.S. Cl. 604/361

(57) ABSTRACT

An absorbent article, such as an incontinence protector, a napkin, a sanitary towel, a panty liner and the like, which includes a liquid-permeable surface layer, which is adapted, during use, to face a user, a liquid-impermeable back side layer and an absorbent body, which is placed between the inner side of the surface layer and the inner side of the back side layer. The absorbent article includes an indicator device which indicates the presence of moisture or liquid in the absorbent article and the indicator device includes at least one first indicator which indicates a first time period during which the indicator device has been exposed to moisture or liquid.

### CROSS-REFERENCE TO RELATED APPLICATIONS

The present application claims the benefit of U.S. Provisional Application Ser. No. 60/524,211, filed in the United States on Nov. 24, 2003, the entire contents of which are hereby explicitly incorporated herein by reference.

### TECHNICAL FIELD

The present invention relates to an absorbent article, such as an incontinence protector, a napkin, a sanitary towel, a panty liner and the like, which comprises a liquid-permeable surface layer, which, during use, is intended to face a user, a liquid-impermeable back side layer and an absorbent body, which is placed between the inner side of the surface layer and the inner side of the back side layer. The absorbent article includes an indicator device which indicates the presence of moisture or liquid in the absorbent article.

### DESCRIPTION OF RELATED ART

The present invention relates to wetness indicators in absorbent articles, for example napkins and incontinence protectors, which are intended for once-only use. In order for these articles to be comfortable and hygienic to wear, it is necessary to replace them at regular intervals once they have become wet. However, a problem associated with this is that it can be difficult to determine whether an article is wet and, in particular, for how long it has been exposed to liquid. The longer the time the article is worn after it has become wet, the greater is the risk that the article will be felt to be uncomfortable to wear and that an unpleasant odor will arise. In addition, the risk of bacteria growing in the article will increase, in turn increasing the risk of infections and irritations arising. It is therefore of the utmost interest, for minimizing these problems, to be able to determine the length of time for which an article has been exposed to liquid.

Wetness indicators in absorbent articles have been described previously. The indicators are usually dyes or chemical substances which appear, or which change color, when wetted. For example, U.S. Pat. No. 4,705,513 describes a napkin which includes an indicator, where color appears through a carrier strip when the napkin becomes wet and which in addition provides information about how wet the napkin is. A problem with U.S. Pat. No. 4,705,513 is that the indicator does not indicate how long the article has been wet, which means, for example, that a person providing care is unable to determine whether it is necessary to change the napkin immediately or whether it is possible to wait with changing the napkin.

U.S. Pat. No. 4,895,567 discloses a wetness indicator in the form of what is termed a “hot-melt” composition, i.e., a lamellar structure which is produced by applying a smelt to a substrate. U.S. Pat. No. 4,895,567 aims to solve the problem by reducing the response time in connection with wetting. It is said to be possible to vary the response time, on exposure to moisture, from a few seconds to a few minutes by varying the composition or its thickness. As was the case in U.S. Pat. No. 4,705,513, the indicator in accordance with U.S. Pat. No. 4,895,567 does not indicate how long the article has been wet, something which in this case, too, means that it is not possible to determine whether a napkin change is imminent or not.

As previously mentioned, it is not satisfactory, from the point of view of hygiene, simply to know whether an absorbent article is wet or not, or to what degree it is wet, since the risk of infections when wearing a wet absorbent article increases with time.

There is consequently a need for an absorbent article which can provide information on the length of time the absorbent article has been exposed to liquid.

### OBJECTS AND SUMMARY

The present invention is directed towards solving the above-specified problems by means of making available an absorbent article which includes an indicator device which provides information regarding the period over which the article has been exposed to liquid, i.e. the length of time which has elapsed since the user has had a discharge of liquid.

Absorbent articles are understood as meaning incontinence protectors, napkins, sanitary towels, panty liners, insertions and the like which comprise an absorbent material and whose principal function is to absorb body liquids, such as urine or blood. An absorbent article of this type comprises a surface layer, which is essentially liquid-permeable and which is intended, during use, to face a user, a liquid-impermeable back side layer and an absorbent body, which is placed between the surface layer and the back side layer. The indicator device is placed in the article and indicates the presence of moisture or liquid. An embodiment of the invention includes a first indicator which indicates a first period of time during which the indicator device has been exposed to moisture or liquid.

When an absorbent article is used, the surface layer is intended to spread the liquid which impacts the article in connection with liquid discharge and to transport it to the absorbent body. In the present case, liquid discharge is intended to mean events which involve a user releasing liquid, for example urine. In addition, the surface layer, together with the back side layer, is intended to encapsulate the absorbent body. The back side layer is intended to function as a barrier layer, i.e. is intended to prevent the leakage of liquid from the absorbent body through the back side layer. In connection with liquid discharge, the liquid is spread through the absorbent body towards the back side layer.

The absorbent body can comprise a number of absorbent layers which possess different spreading abilities and liquid-retaining abilities. One such layer is an upper layer of the absorbent body, which lies closest to the surface layer, and is advantageous in that it spreads the liquid over the extent of the absorbent body and transports the liquid to an underlying layer of the absorbent body which possesses high retention ability. Since the upper layer spreads the liquid more than it retains the liquid, the surface of the surface layer, which abuts the user, remains relatively dry. Corresponding to the upper layer is a lower layer, which has the task of retaining liquid, binding the liquid in its structure and preventing the liquid from being transported back towards the surface layer. However, the lower layer also has a liquid-spreading ability which enables the liquid which impacts the absorbent body to be spread, and distributed essentially uniformly, over time; however, the spreading proceeds more slowly than in the case of the abovementioned upper layer. The lower layer preferably comprises a fiber material in which the liquid is bound in the fiber structure by means of capillary force. Such liquid is henceforth termed capillary liquid. Capillary liquid also arises in other materials which possess structures which allow the liquid to be bound in the structure by means of capillary force. An example of such a material is foam which possesses a structure which comprises cavities or open cells in which liquid can be spread and retained by capillary force.

Capillary liquid gives rise to a vapor phase which is created in the proximal environment and which contains sufficient moisture to give rise to the start of time-keeping by the indicator, i.e. the point in time when the indicator device was initially exposed to moisture or liquid. In those cases in which the absorbent body is unable to store all liquid in its structure, unabsorbed liquid may then impact the indicator device, thereby starting the time-keeping. Unabsorbed liquid can arise, for example, at a time shortly after a liquid discharge, when the liquid impacts a central part of the absorbent article and when the absorbent article has not had time to spread and distribute all the liquid. Another occasion when unabsorbed liquid arises is when the absorbent article is saturated, i.e. when the absorbent article lacks the ability to absorb any more liquid.

The indicator device according to an embodiment of the present invention preferably comprises a number of indicators in the form of microcapsules which contain an envelope layer which encloses an indicator substance. The first indicator thus preferably comprises a microcapsule which contains an indicator substance which is coated with a liquid-soluble first envelope layer. The microcapsule preferably comprises a unit which is essentially spherical and which has a spatial geometry in the form of a sphere, egg or cylinder.

The envelope layer preferably comprises a water-soluble material which dissolves on contact with moisture or liquid and which thereby releases the indicator substance, which then gives an indication. The nature of the liquid-soluble envelope layer determines the time it takes for the envelope layer to dissolve and consequently determines the time which elapses after the liquid discharge before the indicator substance gives a signal. When an envelope material is selected that dissolves after a desired period of time, the indicator device, by releasing the indicator substance, then alerts the user (or, for example, a care provider) that a period has elapsed.

Examples of suitable materials for an envelope layer are water-soluble substances, for example, modified polysaccharides such as hydroxypropylmethyl cellulose, hydroxypropyl cellulose, hydroxyethyl cellulose or polyhyaluronic acid. The latter is particularly suitable in an incontinence product containing an acidic superabsorbent (SAP). Other materials can be alginate and xanthan; proteins such as whey; synthetic polymers such as polyethylene oxide and polyvinyl alcohol; proteins such as pectin; water-soluble polymers, such as polyamines (for example amino acid-based based or amino acrylate-based), polyvinylpyrrolidone, polyglycols, polyphosphates, polyacrylic acid, polymethacrylates or polyphosphazenes, or a combination of the different substances, which can allow for a good start to be obtained when coating or for diluting an indicator substance.

The time taken for the capsules to dissolve can be varied by varying the thickness of the envelope layers, with this resulting in the layers dissolving and releasing the indicator substance, at different rates. Alternatively, the envelope layers can be arranged to possess different crystallinities, for example by using polymers, for example polyethylene oxide or polyvinyl alcohol, which initially possess a given crystallinity. The crystallinity can then be changed by adding different quantities of heat during the drying procedure after the coating. A more crystalline structure is known to have a longer dissolution time. Another example is that the envelope layer can comprise a mixture of hydrophilic and hydrophobic polymers. Varying the quantities of the polymers which are incorporated results in envelopes which exhibit varying tendencies to dissolve on contact with liquid, resulting in envelopes which release the encapsulated indicator substance after different periods of time. In addition, the dissolution time can be varied by altering the molecular weight of the polymers. Long-chain polymers have a longer dissolution time than the shorter polymers in the same polymer group.

It is possible to conceive of several ways of arranging the indicator substance in the microcapsule. The indicator substance can be enclosed within the capsule envelope and be released when the capsule envelope has dissolved. As an alternative, the indicator substance can also be distributed in the capsule envelope itself. The microcapsule can be produced by means of one or more layers of the indicator substance being coated on a core comprising, for example, of a sand granule, a sugar granule, a salt granule or a crystal of the indicator substance, where appropriate mixed with other components such as water-soluble polymers, etc. One or more layers of the material which constitutes the envelope layer is/are then coated on top of the indicator substance. The microcapsules can be manufactured in a fluidizing bed, where the core is allowed to fluidize in a vessel with air such that the core floats freely. The indicator substance is then sprayed into the vessel, for example together with a polymer. After this, an aqueous solution of the substance which is to constitute the envelope layer is then fed in. Finally, the microcapsule which has now been formed is dried using a suitable convective method. The diameter of the microcapsules is preferably from 0.5 μm to 300 μm.

The indicator substance preferably comprises a potent coloring agent which spreads readily in a moist or wet environment. In addition, the coloring agent should preferably be a pharmacologically acceptable dye which is nontoxic and which does not cause allergies in, or irritation to, the wearer and which can be detected readily in the product after it has been released from the microcapsule. Suitable coloring agents are those which are used in the foodstuffs industry and the pharmaceutical industry and in cosmetic preparations. Examples of these substances are to be found in the groups which comprise nitro dyes, monoazo dyes, diazo dyes, phthalocyanine dyes, quinoline dyes, xanthene dyes, triarylmethane dyes, indigoid dyes and vegetable dyes, see, for example, U.S. Pat. No. 3,675,654. Other dyes which are suitable for use in the present invention are iron oxide, chromium oxide (Cr₂O₃, green), manganese violet and ultramarine (synthetic, completely safe dyes which are color-fast). Other suitable dyes include reddish blue, C.I. Pigment Blue 29:77007 [57455-37-5], violet, C.I. Pigment Violet 15:77007 [12769-96-9], pink, C.I. Pigment Red 259:77007 [12769-96-9] (Ullmann's Encyclopedia of Industrial Chemistry, Vol. A24, page 231). Dyes such as methyl red, methyl violet, methyl orange, bromocresol lilac (mauve), ethyl red, bromophenol blue, bromocresol green, crystal violet, cresol red, thymol blue, erythrosin B, 2,4-dinitrophenol, o-cresolphthalein, phenolphthalein, thymolphthalein, alizarin, etc., are also suitable, see, for example, WO 00/76558. The dyes are preferably water-soluble so that any staining of skin or articles of clothing which have come into contact with the dye can easily be removed. The particle size of the dyes is preferably less than the size of the microcapsules in those cases where the indicator substance is coated with an envelope layer. However, certain dyes are of such a type that the dye particles should have a particle size of from 50 μm to 100 μm in order to prevent the particles penetrating into the deeper layers of the epidermis (Umbach, Kosmetik [Cosmetics], Thieme Verlag, Stuttgart 1988). The quantity of the dye in the microcapsule has to be sufficiently large to be able to be detected easily in the article when the relevant period of time has expired, i.e. when the indicator device has been exposed to moisture and/or liquid for the relevant period of time. However, the dyes which are suitable have a very powerful staining effect which means that the quantity of dye in each microcapsule is in the microgram order of size. The indicator substance can also be a chemical substance which changes color when it is exposed to liquid and whose color change can be detected.

According to an embodiment of the invention, the indicator device begins its time-counting when it has been exposed to moisture or liquid. Moisture is understood as meaning a moisture content in the absorbent article which reaches a predetermined level. In this present case, time-counting is understood as meaning the period which starts from the point at which the envelope layer of at least one indicator is exposed to moisture or liquid and consequently begins to dissolve. The envelope layer can, for example, comprise a material which possesses properties which are such that the material begins to dissolve on contact with liquid. The envelope layer can also comprise a material which begins to dissolve on contact with moisture. The predetermined level of the moisture content can consequently be stipulated by the choice of material in the envelope layer. The predetermined level of the moisture content is preferably represented by the moisture content which is obtained in the vapor phase which is formed in connection with the liquid which is retained by capillary force in the absorption layer, as defined above. In this present case, moisture content is consequently understood as being the moisture content which is given by the vapor phase which is formed in connection with the capillary liquid.

In a second embodiment of the invention, the indicator device includes a second indicator which indicates a second period during which the indicator device has been exposed to moisture or liquid. Like the first indicator, the second indicator comprises a coloring agent which has been coated with a liquid-soluble second envelope layer. The second indicator can consequently also comprise microcapsules. The second indicator is preferably arranged to indicate the second period after the first period. An advantage of this embodiment is that a user, or a care-provider, is informed of different time intervals. It can be mentioned, as an example, that the first indicator indicates a first period after three hours by means of the envelope layer then having been at least partially dissolved, in connection with which the indicator substance is released, for example in the form of a green dye. The user/care provider then knows that at least three hours have elapsed since the indicator device was exposed to moisture or liquid, i.e. that three hours have elapsed since the time of the first liquid discharge. Let us say that it is acceptable to allow the napkin to stay in place for six hours. The user/care provider has then been informed that it will soon be time to change the napkin. In this case, the second indicator is expediently arranged to provide an indication after six hours as a result of the second indicator releasing an indicator substance, for example in the form of a red dye, when six hours have elapsed.

The periods which are suitable depend on what condition the user is in. An individual who is suffering from a disease may have a different bacterial flora, making it necessary for the absorbent article to be changed frequently, for example every half hour, every hour or every other hour, etc. By contrast, a healthy patient can be allowed to have a moistened absorbent article for a substantially longer period of time. A crucial factor in calculating periods can consequently be the fear of bacterial growth in different users of differing status. An absorbent article can consequently be equipped with indicator devices which indicate a short period for a certain category of user and indicate a long period for another category of user. The period is determined by the formulation of the different indicators, as described above.

According to one embodiment of the second embodiment of the invention, the indicator device is arranged in the form of a large number of main spherical units in which the second indicator is located behind the first indicator. The spherical unit can have a spatial geometry which is in the form of a sphere, egg or cylinder, or have another suitable appearance. The spherical unit is constructed in such a way that the second indicator contains a core of a second indicator substance which is coated with a second envelope layer. The first indicator is then constructed after that by the first indicator substance being coated over the second envelope layer, after which the first envelope layer is coated over the first indicator substance.

When the indicator device has been exposed to moisture or liquid, the first envelope layer begins to dissolve and, after the predetermined first period of time, at least parts of the first envelope layer have been dissolved such that the first indicator substance is released. After the first indicator substance has been released, the second envelope layer is exposed to moisture or liquid and consequently begins to dissolve. After a predetermined second period of time, the second envelope layer has been dissolved and the second indicator substance is released. In order to obtain the effect which was specified in the above example, the first envelope layer is dissolved after three hours and the green indicator substance then indicates the first period of three hours in length. Subsequently, the second indicator layer dissolves after a further three hours and the red indicator substance is released. The red indicator substance is consequently released six hours after the spherical unit has been exposed to moisture or liquid. The first period of time can be longer than the second period of time, or of the same length or shorter, depending on the desired intervals.

According to another embodiment of the second embodiment of the invention, the indicator device is arranged in such a way that the first indicator and the second indicator comprise separate units which are separated from each other. In this case, the first indicator and the second indicator comprise microcapsules where the first envelope layer is dissolved at a different rate from the envelope layer in the second indicator. In accordance with the example above, the first indicator would be equipped with a first envelope layer which is dissolved three hours after contact with moisture or liquid. The second indicator would then be equipped with a second envelope layer which is dissolved six hours after contact with moisture or liquid. The differences in dissolution time between the first envelope layer and the second envelope layer can be achieved using one of the abovementioned methods, for example using envelope layers which are composed of the same material but are of different thicknesses, or using envelope layers which are composed of different materials.

In another embodiment of the invention, additional indicators can be included in the indicator device. The additional indicators can then represent additional time intervals. The manufacturer can consequently choose to manufacture an absorbent article which includes an indicator which indicates wetness only for one period of time. In addition to this, the manufacturer can choose a number of indicators which represent periods of different length, thereby providing the user with information about different time intervals, i.e. sequential chronological information about the length of time for which the indicator device has been exposed to moisture or liquid.

The different indicators preferably contain indicator substances which give different signals on different occasions. The indicator substances can comprise different dyes. However, an alternative is that the first indicator emits an indicator substance in the form of a dye and the next indicator in chronological order then releases a substance which changes the color of, or neutralizes, the first-mentioned indicator substance, for example by means of a change in acidity or by means of a chemical reaction.

According to one embodiment of the invention, the first indicator contains a liquid-absorbing absorption material which is attached to the first envelope layer. In an analogous manner, the second indicator can also contain a liquid-absorbing absorption material which is attached to the second envelope layer. An absorption material of this nature can, for example, be a superabsorbent (SAP). Superabsorbents are chemical substances which absorb liquid while forming a gel.

In all the abovementioned embodiments of the invention, the indicator is preferably located in the absorbent body in such a way that the indication provided by the indicator is visible, to an individual, on the exterior of the back side layer, through the back side layer. The back side layer is therefore preferably designed with a transparent or translucent section which is connected to the indicator device. Naturally, the whole of the back side layer can be transparent or translucent. The crucial point is that the back side layer should allow the indicator substance to be visible/detectable for a user or a care-provider. In one embodiment of the invention, the indicator devices are arranged in connection with both the back side layer and the absorbent body. In one embodiment, the indicator particles are not arranged directly against the back side layer but are instead located at expedient sites in the absorbent body. The absorbent body has then to be designed in such a way that the indicator substance is visible through the back side layer, for example by means of the absorbent body allowing the indicator substance to migrate towards the back side layer.

The indicator device can consequently be located anywhere in the absorbent article where the indicator substance is readily visible. This means that the indicator device can be concentrated in one region of the said article or spread over the whole of the product. If desired, the indicator device can form a pattern or a text. The present invention also allows the length of wetting time before the indicator device is to give a signal to be matched to personal requirements. This can be achieved by manufacturing products which contain indicator devices which give signals after predetermined periods of time.

So as to ensure that the indicator device functions satisfactorily, it is preferably exposed to moisture or liquid concomitantly with liquid discharge. Consequently, the indicator device is preferably located such that it is rapidly reached by liquid or moisture when the latter penetrates into the product. The part of the absorbent body which lies closest to the genitals of a user is termed the wetting point. In the wetting point, transport of liquid through the absorbent body to the back side layer takes place even in connection with a relatively minor discharge of liquid. The indicator device according to the present invention should consequently be located in the absorbent article below the wetting point and adjacent to the back side layer or in the vicinity of the back side layer. The indicator device should be located in the article in such a way that the indicator substance is visible through the back side layer when liquid is being indicated. It is possible to facilitate the transport of liquid through the article by using wicks. Wicks comprise a material which possesses very good liquid transport properties and which is located in the absorbent body as a liquid conductor. The wicks are expediently located with one end in the vicinity of the surface layer and one end in the vicinity of the back side layer. In addition, the absorbent body can be designed with channels which transport liquid in the direction from the surface layer towards the back side layer, see, for example, PCT/SE02/01494. The indicator device is then expediently located adjacent to the channels and adjacent to the back side layer.

When the absorbent article is being manufactured, the microcapsules can be attached to a substrate which is applied in the article. In addition, the microcapsules can be applied directly against the inner side of the back side layer or against the absorbent body. In addition, the absorbent article can be fitted with a spreading layer against the inner side of the back side layer. The indicator devices can then be located adjacent to the spreading layer and the absorbent body. The spreading layer spreads the liquid, which reaches the spreading layer, along the back side layer. The spreading layer makes it possible for liquid to reach a large number of indicator devices, thereby affording the advantage that a large number of indicator devices indicate liquid essentially simultaneously, resulting in a strong signal. Another advantage of the spreading layer is that the indicator substance is spread in the spreading layer, resulting in a clear signal.

The liquid-blocking back side layer comprises a liquid-impermeable material. While thin, liquid-tight plastic films are suitable for the purpose, it is also possible to use material which was originally liquid-permeable but which has been provided with a lining composed of plastic, resin or some other liquid-tight material. This thereby prevents leakage of liquid from the underside of the absorbent article. The blocking layer can consequently comprise any material which meets the criterion of liquid impermeability and which exhibits flexibility and kindness to the skin which is adequate for the purpose. Examples of materials which are suitable for use as blocking layers are plastic films, nonwoven fibers and laminates thereof. The plastic film can, for example, be composed of polyethene, polypropene or polyester. The blocking layer can alternatively comprise a laminate of a liquid-impermeable plastic layer, which faces the absorbent body, and a nonwoven, which faces the underclothes of the user. Such a construction provides a leakage-secure blocking layer which has a textile feel. The liquid-blocking back side layer can also comprise a vapor-permeable material. Such a breathable back side layer can, for example, be composed of what is termed SMS (spunbond-meltblown-spunbond) material or a breathable plastic film comprising polyethene. A plastic film of this nature is described in EP 283 200. In order to preserve the breathability even when the material has been applied to a product, the underside of the product should not be entirely covered by attachment means.

The surface layer can be composed of any conventional material, for example a nonwoven or a perforated plastic film or a laminate of a perforated plastic film and a nonwoven.

The absorbent body is expediently produced from one or more layers of cellulose pulp. The pulp can originally be present in rolls, bales or sheets which, in connection with manufacturing the sanitary towel, are dry-defibered and converted in fluffed form into a pulp mat, sometimes with what are termed superabsorbents, which are polymers which have the ability to absorb several times their own weight of water or body fluid, being mixed in. An alternative to this is to dry-form a pulp mat as described in WO 94/10956. Examples of other absorbent materials which can be used are different types of natural fibers such as cotton fibers, peat, or the like. It is naturally also possible to use absorbent synthetic fibers or particles of a highly absorbent polymeric material of the type which, in connection with absorption, chemically binds large quantities of liquid, with the formation of a liquid-containing gel, or mixtures of natural fibers and synthetic fibers. In addition, the absorbent body can contain other components such as form-stabilizing elements, liquid-spreading elements or binders, such as thermoplastic fibers which have been heat-treated in order to hold short fibers and particles together to form a continuous unit. It is also possible to use different types of absorbent foam material in the absorbent body.

An indicator according to an embodiment of the present invention could be used in an absorbent particle which is in the form of a tampon. One reason for using an indicator device in accordance with the present invention in a tampon is to teach the user time limits for how long a tampon should be used. Thus, the indicator device indicates that the tampon has been used, i.e. has been exposed to moisture or liquid, for a certain predetermined period which specifies the maximum time the tampon can be used. In addition, the tampon can be equipped with an indicator device which specifies several time intervals with, for example, the first time interval specifying a time which is too short, a second time interval specifying a period which is appropriate and a third time interval specifying a time of use which is too long. The indicator device does not need, in this present case, to be arranged directly at the surface layer of the tampon but can be arranged in the absorbent material of the tampon such that the indicator substance is visible when the tampon is removed from the user's vagina.

### BRIEF DESCRIPTION OF THE DRAWINGS

The invention will be described below in connection with a number of figures where:

FIG. 1 schematically shows a perspective view, from the back, of an absorbent article according to an embodiment of the invention in the opened-out state;

FIG. 2 schematically shows a section of an indicator in an indicator device in accordance with a first embodiment of the invention;

FIG. 3 schematically shows a sectional view, in the section A-A in FIG. 1, in accordance with one embodiment of the indicator device shown in FIG. 2;

FIG. 4 schematically shows a sectional view, in the section A-A in FIG. 1, in accordance with a second embodiment of the indicator device shown in FIG. 2;

FIG. 5 schematically shows a sectional view, in the section A-A in FIG. 1, in accordance with yet another embodiment of the indicator device shown in FIG. 2;

FIG. 6 schematically shows a section of a microcapsule in an indicator device in accordance with a second embodiment of the invention, and where;

FIG. 7 schematically shows a sectional view, in the section A-A in FIG. 1, using an indicator device as shown in FIG. 6.

### DETAILED DESCRIPTION OF THE PREFERRED EMBODIMENTS

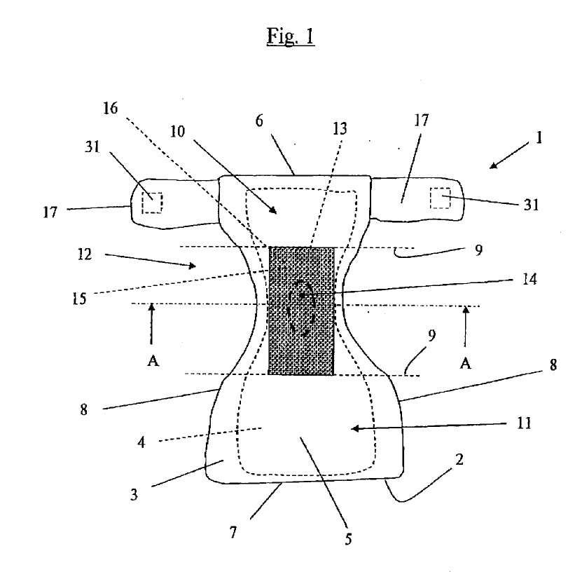

FIG. 1 schematically shows a perspective view of an absorbent article 1 in the opened-out state. The article 1 comprises a surface layer 2, a back side layer 3 and, between them, an absorbent body 4. FIG. 1 shows the absorbent article in a view from the back, i.e. towards the outside 5 of the back side layer. The absorbent article 1 has a longitudinal extension between two short sides 6, 7, and a lateral extension between two long sides 8. In addition, the absorbent article 1 is divided, in the longitudinal direction, into two end sections 10, 11, and a central section 12 which extends between them, by means of two broken lines 9 which extend parallel to each other. The division into sections is not to be interpreted literally but is solely an aid when describing the invention. In practice, the borders between the different sections 10, 11 and 12 comprise transition zones.

FIG. 1 shows an indicator device 13 according to an embodiment of the invention which is located between the back side layer 3 and the absorbent body 4. In FIG. 1, the indicator device 13 is located at the central section 12 of the absorbent article 1, preferably in a position which at least partially coincides with a wetting point 14 of the absorbent article 1. FIG. 1 shows that the indicator device 13 contains a number of indicators 15. FIG. 1 shows the back side layer 3 as being transparent, for which reason both the indicator device 13 and the absorbent body 4 can be clearly seen. FIG. 1 shows the indicator device 13 as being a rectangular unit. The indicator device 13 can naturally be designed to have another geometry, for example oval, hour-glass shaped, square, round. In addition, the indicator device 1 can be applied along the whole of the extension of the absorbent article 1 or only the extension of the absorbent body 4. The indicator device 13 comprises a large number of indicators 15 in the form of microcapsules 16.

In addition, FIG. 1 shows two side sections 17 which extend laterally and which are attached to the one end section 10. When the article 1 is being used, the article 1 is bent around the crotch part of a user in such a way that the one end section 11 is placed against the lower part of the abdomen of the user and the other end section 10 is placed against the lower part of the back of the user. When the article 1 is being used, the side sections 17 are intended to be bent over the hip part of the user and, after that, to be attached, via attachment means 31, to the opposite end section 11. The article 1 is designed in such a way that, when the article 1 is being used, the genitals of the user are located in the central section 12, with the wetting point 14 also coming to lie in the central section 12.

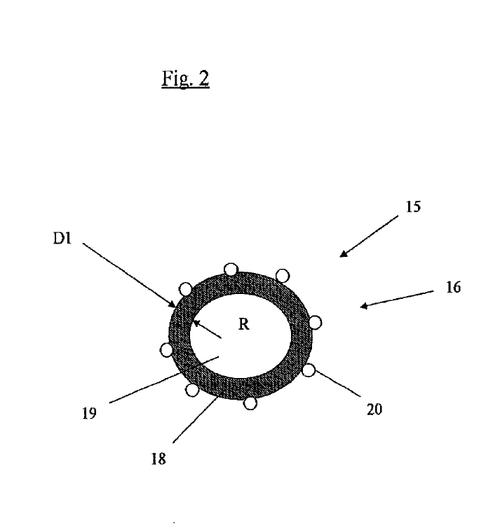

FIG. 2 schematically shows a section of an indicator 15 in an indicator device 13 in accordance with a first embodiment of the invention. FIG. 2 shows the indicator device as a microcapsule 16 which has an oval cross section. The microcapsule 16 can thus be essentially cylindrical, spherical or ovoid. The microcapsule 16 comprises a first envelope layer 18 and a first indicator substance 19, with the indicator substance 19 in FIG. 2 being marked by the letter “R”. The first envelope layer 18 has a certain thickness D1 and comprises one of the materials which were enumerated previously. FIG. 2 shows that absorbent particles 20 are attached to the envelope layer 18. The absorbent particles 20 are intended to absorb liquid, with this being meant to afford the advantage of the envelope layer 18 being rapidly, and after that continuously, exposed to moisture or liquid, with this in turn giving rise to the desired time-measurement. The absorbent particles 20 are not necessary but simply constitute one embodiment of the invention. The indicator 15 can consequently consist solely of a microcapsule 16 which contains an envelope layer 18 which surrounds an indicator substance 19.

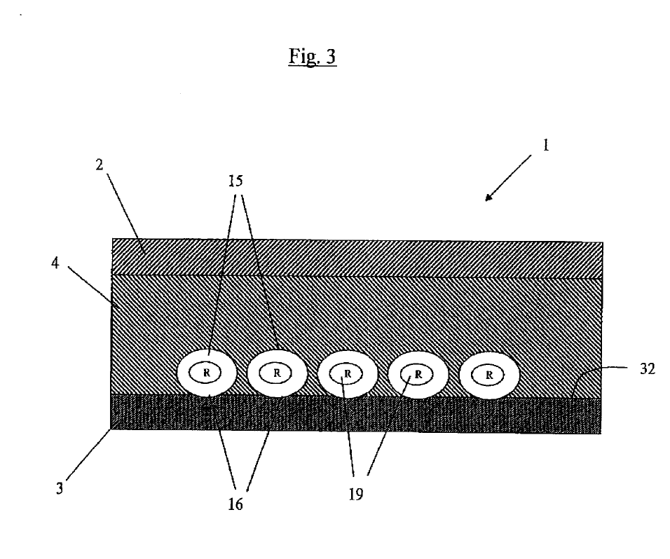

FIG. 3 schematically shows a sectional view in the section A-A in FIG. 1, in accordance with one embodiment of the indicator device 13 shown in FIG. 2. For reasons of clarity, the figure only shows five microcapsules 16. The number can naturally be increased many times over. All the microcapsules 16 in FIG. 3 are of the same type, i.e. they all possess an envelope layer 18 exhibiting the same dissolution time; furthermore, the microcapsules 16 all release the same type of indicator substance 19. Since the indicators 15 have the same dissolution time, the embodiment shown in FIG. 3 is consequently suitable for measuring a period of time. The microcapsules 16 are preferably arranged in the absorbent body 4 towards the inner side 32 of the back side layer 3.

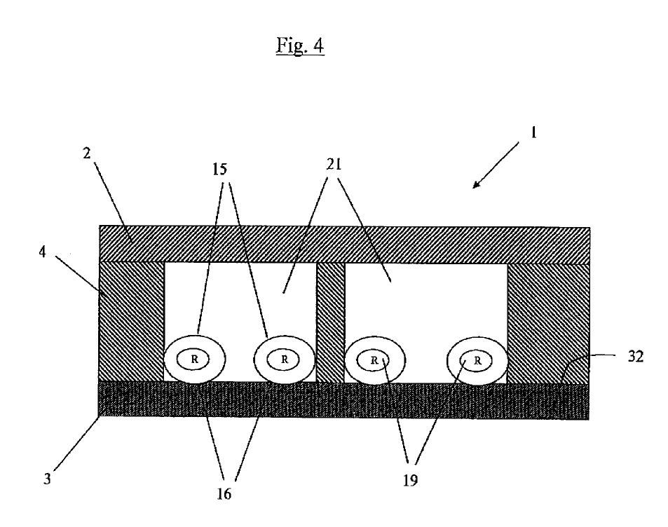

FIG. 4 schematically shows a sectional view in the section A-A in FIG. 1 in accordance with another embodiment of the indicator device 13 shown in FIG. 2. The microcapsules 16 shown in FIG. 4 correspond to the microcapsules 16 which are shown in FIG. 3. FIG. 4 shows that the microcapsules 16 are arranged at channels 21 which are present in the absorbent body 4. The channels 21 provide the liquid with more rapid transport from the surface layer 2 to the indicator device 13 than if the liquid had been transported through an absorbent body 4 without any channels 21. FIG. 4 naturally does not show channels in cross section which are to scale; instead, for reasons of clarity, the figure only shows two channels with two indicators 15 adjacent to each channel 21. FIG. 4 can consequently be seen as an isolated minor part of the absorbent article, merely showing a minor subset of all the channels and indicators which can be present in the absorbent article.

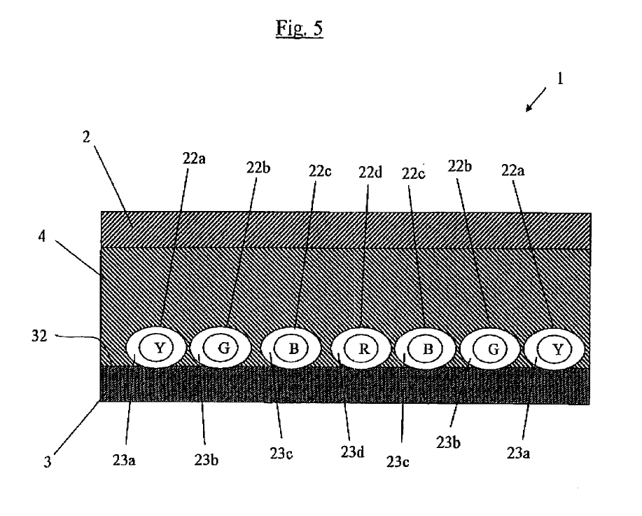

FIG. 5 schematically shows a sectional view in the section A-A in FIG. 1 in accordance with yet another embodiment of the indicator device 13 shown in FIG. 2. In this case, the indicator device 13 contains four different indicators 22a-d possessing different indicator substances marked with the letters Y, G, B and R. The letters Y, G, B and R can, for example, indicate four different colours. The different indicators 22a-d possess envelope layers which exhibit different dissolution times, resulting in the indicators 22a-d releasing the indicator substances Y, G, B and R at different times when the different indicators 22a-d have been simultaneously exposed to moisture or liquid. The embodiment shown in FIG. 5 is suitable for sequential chronological indication, i.e.:
the first indicator 22a possesses a first envelope layer 23a which is dissolved after a first time and which then gives a signal by releasing the first indicator substance Y;
the second indicator 22b possesses a second envelope layer 23b which is dissolved after a second time and which then gives a signal by releasing a second indicator substance G;
the third indicator 22c possesses a third envelope layer 23c which is dissolved after a third time and which then gives a signal by releasing a third indicator substance B;

the fourth indicator 22d possesses a fourth envelope layer 23d which is dissolved after a fourth time and which then gives a signal by releasing a fourth indicator substance R.

The periods of time are in ascending chronological order, i.e. the first period of time is shorter than the second, etc.

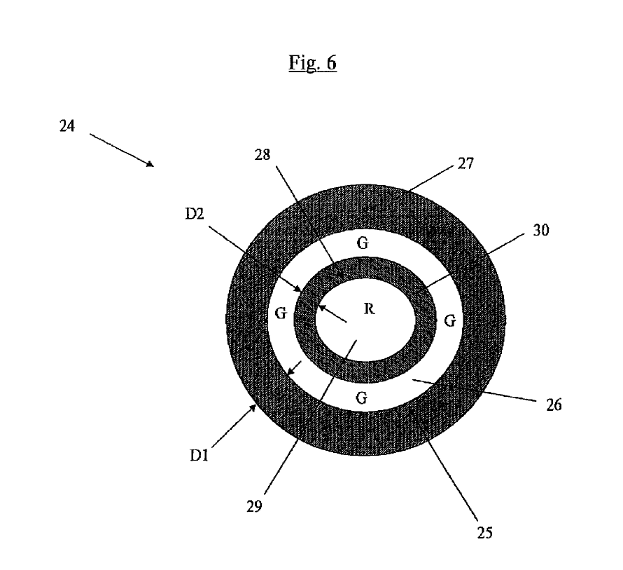

FIG. 6 schematically shows a section of a microcapsule 24 in an indicator device 13 in accordance with a second embodiment of the invention. The microcapsule 24 possesses an essentially circular cross section, which means that the microcapsule 24 can be spherical, ovoid or cylindrical. Other spatial geometries can also occur, for example cubic microcapsules. In this present case, the microcapsule 24 contains two indicators 25, 28 in a layered structure. The second indicator 28 comprises a core of a second indicator substance 29 (also marked with the letter R) which is coated with a second envelope layer 30. The first indicator 25 comprises a first indicator substance 26 (also marked with the letter G), which is coated on the second envelope layer 30, and a first envelope layer 27 which is coated on the first indicator substance 26.

At least part of the first envelope layer 27 is entirely dissolved after a first time and then gives a signal by releasing the first indicator substance 26. Subsequently, the second envelope layer 30 begins to dissolve and, after a second time, at least parts of the second envelope layer 30 have dissolved completely and then give a signal by releasing the second indicator substance 29. FIG. 6 shows the first envelope layer 27 having a thickness D1 and the second envelope layer having a thickness D2. When the first envelope layer comprises the same material as the second envelope layer, the dissolution times can be varied by varying the thicknesses D1 and D2; the greater the thickness of the envelope layer, the longer is its dissolution time. In other cases, the dissolution times can be varied by manufacturing the first envelope layer 27 in a material which is different from that used in the second envelope layer 30.

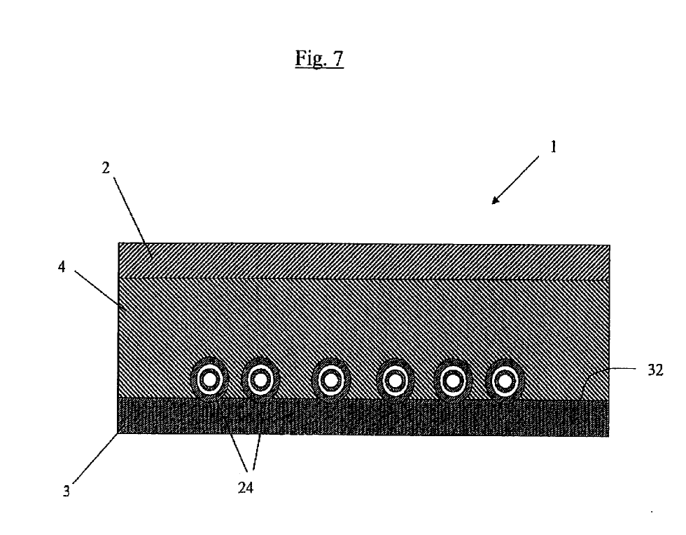

FIG. 7 schematically shows a sectional view in the section A-A in FIG. 1, using an embodiment of the indicator device 13 as shown in FIG. 6. The microcapsules 24 are placed in the absorbent body 4 and towards the inner side 32 of the back side layer 3. The microcapsules 24 shown in FIG. 7 can also be placed adjacent to channels 21 like those shown in FIG. 4.

The invention is not limited to the embodiments described above but can be varied within the scope of the subsequent patent claims. As an example, it can be mentioned that the indicator devices can be arranged adjacent to the back side layer such that a spreading layer is placed between the back side layer and the indicator devices. The spreading layer spreads the liquid in the article to the different indicator devices.

Although only preferred embodiments are specifically illustrated and described herein, it will be appreciated that many modifications and variations of the present invention are possible in light of the above teachings and within the purview of the appended claims without departing from the spirit and intended scope of the invention.

1. An absorbent article comprising a liquid-permeable surface layer, which is adapted, during use, to face a user, an essentially liquid-impermeable back side layer and an absorbent body, which is placed between the surface layer and the back side layer, with the absorbent article including an indicator device which indicates a presence of moisture or liquid in the absorbent article, wherein the indicator device contains at least one first indicator which indicates a first time period during which the indicator device has been exposed to moisture or liquid.

2. The absorbent article according to claim 1, wherein the first indicator comprises a first indicator substance which is coated with a liquid-soluble first envelope layer.

3. The absorbent article according to claim 2, wherein the first indicator substance comprises a dye.

4. The absorbent article according to claim 1, wherein the indicator device begins to dissolve when the first indicator has been exposed to a moisture content in the absorbent article reaching a predetermined level, with counting of the first time period then starting.

5. The absorbent article according to claim 4, wherein the predetermined level corresponds to a vapor-phase which is formed from liquid which has been retained in the absorbent body by capillary force.

6. The absorbent article according to claim 1, wherein the indicator device contains a second indicator which indicates a second period during which the indicator device has been exposed to moisture or liquid.

7. The absorbent article according to claim 6, wherein the second indicator is arranged to indicate the second period of time after the first period of time.

8. The absorbent article according to claim 6, wherein the indicator device is arranged in a form of what is an essentially spherical unit, with the second indicator being placed under the first indicator.

9. The absorbent article according to claim 6, wherein the first indicator and the second indicator comprise separate units which are separated from each other.

10. The absorbent article according to claim 6, wherein the second indicator contains a second indicator substance which is coated with a liquid-soluble second envelope layer.

11. The absorbent article according to claim 1, wherein the first indicator contains a liquid-absorbing absorbent material which is attached to a first envelope layer.

12. The absorbent article according to claim 1, wherein the indicator device is placed in the absorbent article such that an indication of the indicator device is visible to an individual on an outside of the back side layer, through the back side layer.

13. The absorbent article according to claim 1, wherein the indicator device is placed adjacent to the back side layer.

14. The absorbent article according to claim 1, wherein the back side layer is designed to have a transparent section adjacent to the indicator device.
